(19) United States
(12) Patent Application Publication
(10) Pub. No.: US 2025/0002616 A1
(43) Pub. Date: Jan. 2, 2025

(54) Systems and Methods for Alkene Oligomerization

(71) Applicant: California Institute of Technology, Pasadena, CA (US)

(72) Inventor: Emmanuelle Despagnet-Ayoub, Pasadena, CA (US)

(73) Assignee: California Institute of Technology, Pasadena, CA (US)

(21) Appl. No.: 18/749,292

(22) Filed: Jun. 20, 2024

Related U.S. Application Data
(60) Provisional application No. 63/521,925, filed on Jun. 20, 2023.

(51) Int. Cl. C08F 4/02; C08F 4/80; C08F 110/14

(52) U.S. Cl. CPC C08F 4/02; C08F 4/80; C08F 110/14; C08F 2410/04

(57) ABSTRACT

Systems and methods for multifunctional catalyst systems supported on carbon nanotubes (CNTs) for olefin oligomerization are described. The catalyst systems can directly convert CO₂ to jet-range (C12-C18) hydrocarbons. This conversion can be achieved by a cooperative, tandem catalyst system supported on CNTs converting CO₂ to olefins (C2-C9) with the catalyst systems followed by oligomerization.

### CROSS-REFERENCE TO RELATED APPLICATIONS

The current application claims the benefit of and priority under 35 U.S.C. § 119 (e) to U.S. Provisional Patent Application No. 63/521,925 entitled “Alkene Oligomerization with Transition Metal Catalysts Supported on Carbon-Nanotubes” filed Jun. 20, 2023. The disclosure of U.S. Provisional Patent Application No. 63/521,925 is hereby incorporated by reference in its entirety for all purposes.

### STATEMENT OF FEDERAL FUNDING

This invention was made with government support under Grant No. 80NMO0018D0004 awarded by NASA (JPL). The government has certain rights in the invention.

### FIELD OF THE INVENTION

The present invention generally relates to systems and methods for alkene oligomerization with catalysts supported on carbon nanotubes.

### BACKGROUND OF THE INVENTION

In organic chemistry, an alkene, or olefin, is a hydrocarbon containing a carbon-carbon double bond. The double bond may be internal or in the terminal position. Terminal alkenes are also known as α-olefins. Higher alkenes are intermediate products in the manufacture of hydrocarbon products such as solvents, higher alcohols, aldehydes and acids. They can be produced by oligomerization of source materials containing alkenes with 2 to 12 carbon atoms by contacting the source materials with oligomerization catalysts.

### BRIEF SUMMARY OF THE INVENTION

Many embodiments are directed to systems and methods for oligomerization of alkenes with 3 or more carbon atoms using catalysts supported on carbon nanotubes.

Some embodiments include a catalyst system for alkene oligomerization, comprising: a catalyst complex supported on a plurality of carbon nanotubes (CNTs), wherein the catalyst complex comprises a transition metal; wherein the catalyst system catalyzes an alkene oligomerization reaction, wherein an alkene with at least 3 carbon atoms is polymerized to form a product such that a number of carbon atoms of the product is at least doubled.

In some embodiments, the transition metal is selected from the group consisting of: iron, nickel, and chromium.

In some embodiments, at least one of the plurality of CNTs is a single-walled carbon nanotube or a multi-walled carbon nanotube.

In some embodiments, the alkene is selected from the group consisting of: propylene, 1-butene, 2-butene, isobutylene, 1-pentene, 2-pentene, 2-methyl-1-butene, 3-methyl-1-butene, 2-methyl-2-butene, 1-hexene, 2-hexene, 3-hexene, 2-methyl-1-pentene, 3-methyl-1-pentene, 4-methyl-1-pentene, 2-methyl-2-pentene, 3-methyl-2-pentene, 4-methyl-2-pentene, 2,3-dimethyl-1-butene, 3,3-dimethyl-1-butene, 2,3-dimethyl-2-butene, and 2-ethyl-1-butene.

In some embodiments, the alkene oligomerization is a dimerization reaction, and the number of carbon atoms of the product is doubled.

In some embodiments, the alkene oligomerization is a trimerization reaction, and the number of carbon atoms of the product is tripled.

In some embodiments, the catalyst complex is supported on a plurality of CNTs via a method selected from the group consisting of: a covalent bond, an electrostatic bond, and π-π stacking.

In some embodiments, the catalyst complex forms at least one amid bond with at least one of the plurality of CNTs.

Some embodiments further comprise a co-catalyst.

In some embodiments, the co-catalyst is selected from the group consisting of: methyl aluminoxane, modified methyl aluminoxane, diethylaluminium chloride (Et2AlCl), and EtAlCl2.

In some embodiments, the alkene is 1-hexene, the catalyst complex comprises iron, and the product has 12 carbon atoms.

In some embodiments, the product is configured to be a portion of jet fuel.

Some embodiments include a method for alkene oligomerization, comprising: catalyzing an alkene oligomerization reaction with a catalyst system comprising a catalyst complex supported on a plurality of carbon nanotubes (CNTs), wherein an alkene with at least 3 carbon atoms is polymerized to a product such that a number of carbon atoms of the product is at least doubled.

In some embodiments, the catalyst complex comprises a transition metal.

In some embodiments, the transition metal is selected from the group consisting of: iron, nickel, and chromium.

In some embodiments, at least one of the plurality of CNTs is a single-walled carbon nanotube or a multi-walled carbon nanotube.

In some embodiments, the alkene is selected from the group consisting of: propylene, 1-butene, 2-butene, isobutylene, 1-pentene, 2-pentene, 2-methyl-1-butene, 3-methyl-1-butene, 2-methyl-2-butene, 1-hexene, 2-hexene, 3-hexene, 2-methyl-1-pentene, 3-methyl-1-pentene, 4-methyl-1-pentene, 2-methyl-2-pentene, 3-methyl-2-pentene, 4-methyl-2-pentene, 2,3-dimethyl-1-butene, 3,3-dimethyl-1-butene, 2,3-dimethyl-2-butene, and 2-ethyl-1-butene.

In some embodiments, the alkene oligomerization is a dimerization reaction, and the number of carbon atoms of the product is doubled.

In some embodiments, the alkene oligomerization is a trimerization reaction, and the number of carbon atoms of the product is tripled.

In some embodiments, the catalyst complex is supported on a plurality of CNTs via a method selected from the group consisting of: a covalent bond, an electrostatic bond, and π-π stacking.

In some embodiments, the catalyst complex forms at least one amid bond with at least one of the plurality of CNTs.

In some embodiments, the catalyst system further comprises a co-catalyst.

In some embodiments, the co-catalyst is selected from the group consisting of: methyl aluminoxane, modified methyl aluminoxane, diethylaluminium chloride (Et2AlCl), and EtAlCl2.

In some embodiments, the alkene is 1-hexene, the catalyst complex comprises iron, and the product has 12 carbon atoms.

In some embodiments, the product is configured to be a portion of jet fuel.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as exemplary embodiments of the invention and should not be construed as a complete recitation of the scope of the invention. It should be noted that the patent or application file contains at least one drawing executed in color. Copies of this patent or patent application publication with color drawing(s) will be provided by the Office upon request and payment of the necessary fee.

FIG. 1 illustrates various anchoring modes using CNTs support in accordance with an embodiment.

FIG. 2 illustrates a synthesis scheme of the heterogenized iron catalyst on CNTs in accordance with an embodiment.

FIG. 3 illustrates catalyst complex loading on MWCNTs in accordance with an embodiment.

FIG. 4 illustrates TEM/EDS of immobilized on CNTs in accordance with an embodiment.

FIG. 5 illustrates heterogenized iron catalysts on CNTs in accordance with an embodiment.

FIG. 6A illustrates GC trace of 1-hexene oligomers in accordance with an embodiment.

FIG. 6B illustrates 1H NMR of 1-hexene oligomers in accordance with an embodiment.

FIGS. 7A and 7B illustrate chemical structures of catalysts for heterogenization in accordance with an embodiment.

FIG. 8 illustrates UV-Vis of the nickel complexes A1, B1, and B11 in accordance with an embodiment.

FIG. 9A illustrates the chemical structure of the Cr catalyst complex in accordance with an embodiment.

FIG. 9B illustrates a schematic of heterogenization of the Cr catalyst complex in accordance with an embodiment.

### DETAILED DESCRIPTION OF THE INVENTION

Homogeneous liquid-phase alkene oligomerization encounters challenges with product separation due to the formation of liquid oligomers and issues with catalyst recyclability. Hence, there is a growing interest in heterogeneous catalysts. Metal-organic frameworks (MOFs), zeolites, and mesoporous silica are commonly employed as supports for olefin oligomerization. However, these heterogenized catalysts often exhibit diminished activity and require high operating temperatures.

Supported catalysts for ethylene polymerization and oligomerization have gathered great interest due to the benefits of catalyst recyclability and ease of removal from reaction media. However, previous work focused on the oligomerization of ethylene instead of heavy olefins. (See, e.g., S. Talebnezhad, et al., Colloid Polym Sci, 293, 721-733, 2015; L. Zhang, et al., Catalysis Today, 235, 15, 33-40, 2014; S. M. Alshehri, et al., Advances in Polymer Technology, 35, 1, 2016; A. Peng, et al., Catalysis, 14, 268, 2024; the disclosures of which are incorporated by reference.) This heterogenization process can involve immobilizing an efficient homogeneous catalyst onto a selected support such as zeolites, silica, metal-organic frameworks (MOFs), or covalent-organic frameworks (COFs). Carbon nanotubes (CNTs) can be a promising support platform due to their high mechanical strength and low solubility in organic solvents.

Many embodiments provide oligomerization of olefin with three or more carbon atoms using catalysts supported on carbon nanotubes. Several embodiments provide heterogenization of oligomerization catalysts with ligands (organic frameworks) and transition metals such as (but not limited to) iron, nickel, and/or chromium. The catalysts can be selected for the desired oligomerization reactions. In some embodiments, the catalysts are supported on CNTs. Some embodiments use iron complex supported on CNTs to catalyze olefin oligomerization. Some embodiments use nickel complex supported on CNTs to catalyze olefin oligomerization. Some embodiments use chromium complex supported on CNTs to catalyze olefin oligomerization. The catalyst complex can be bound to the CNTs in various methods such as (but not limited to) covalent bonding, electrostatic bonding, and/or π-π stacking in accordance with some embodiments. Certain embodiments bind catalysts to CNTs using amide bonds. Certain embodiments bind catalysts to CNTs via Van der Waals forces with aryl substituents. In many embodiments, various types of CNTs such as (but not limited to) single-walled CNTs (SWCNTs) and multi-walled CNTs (MWCNTs) can be used as support for the catalyst complex.

In various embodiments, the olefins with three or more carbon atoms are also referred to as heavy olefins. Many embodiments use the catalyst complex supported on CNTs for oligomerization of C3 alkenes, or C4 alkenes, or C5 alkenes, or C6 alkenes, or C7 alkenes, or C8 alkenes, or C9 alkenes, or C10 alkenes, or C11 alkenes, or C12 alkenes, or C13 alkenes, or C14 alkenes, or C15 alkenes, or C16 alkenes, or C17 alkenes, or C18 alkenes, or C19 alkenes, or C20 alkenes. Examples of the heavy olefins include (but are not limited to) propylene, butene, butene isomers, 1-butene, 2-butene, isobutylene, pentene, pentene isomers, 1-pentene, 2-pentene, 2-methyl-1-butene, 3-methyl-1-butene, 2-methyl-2-butene, hexene, hexene isomers, 1-hexene, 2-hexene, 3-hexene, 2-methyl-1-pentene, 3-methyl-1-pentene, 4-methyl-1-pentene, 2-methyl-2-pentene, 3-methyl-2-pentene, 4-methyl-2-pentene, 2,3-dimethyl-1-butene, 3,3-dimethyl-1-butene, 2,3-dimethyl-2-butene, 2-ethyl-1-butene.

The catalyst systems in accordance with many embodiments have the potential to convert heavy olefins into fuel (heavier olefins). This could have benefits for providing propellant for the spacecraft ascent vehicles and may also facilitate advanced exploration missions by establishing a long-term refueling network infrastructure on other locations beside Earth. Each kilogram of propellant produced locally (not on Earth) can potentially save substantial mass. Thus, the leverage afforded by in situ production of gasoline fuel as a propellant underscores its significant benefits in reducing launch mass for any mission.

Several embodiments provide synthesis processes for these catalyst complex supported on CNTs. Some embodiments modify the transition metal complex to achieve optimal binding with the CNT support via ligand design before being heterogenized. Following thorough characterization of the heterogeneous catalyst, the system can be activated with a co-catalyst such as (but not limited to) modified methylaluminoxane (MMAO) for alkene oligomerization

Some embodiments provide the heterogenization of a pyridine bis-imine iron complex on CNTs and its activity in the dimerization of 1-hexene. The CNT/iron complex can be synthesized using imido linkage and characterized by TEM/EDS and ICP-OES to determine the distribution and loading of the catalyst. An optimal iron: CNT ratio can be about 1:1. The system exhibits high reactivity in the dimerization of 1-hexene, achieving about 94% olefin consumption and about 65% C12 formation. The CNT-supported catalysts in accordance with many embodiments can operate via a mechanism similar to that of the homogeneous catalyst.

### CNT-Supported Iron Complex Catalyzes 1-Hexene Dimerization

The heterogenization of catalysts has advantages such as catalyst recyclability and easy removal from reaction media. This strategy often involves anchoring an efficient homogeneous catalyst onto a support through ligand framework modification. One application of this supported catalyst is in olefin polymerization, addressing the demand for environmentally friendly processes through catalyst recyclability. Research has been conducted, with supports including zeolites, silica, MOFs, and COFs. CNTs offer desired properties like high mechanical strength and low solubility in organic solvents. The development of functionalized CNTs has broadened their catalytic applications. Various anchoring strategies have been reported, including electrostatic anchoring with methyl aluminoxane (MAO) on the surface, covalent attachment often through an amide linkage, and π-π stacking between the aromatic framework of the ligand and the nanotubes. The covalent system may provide a more stable structure with a strong bond between the support and the catalyst.

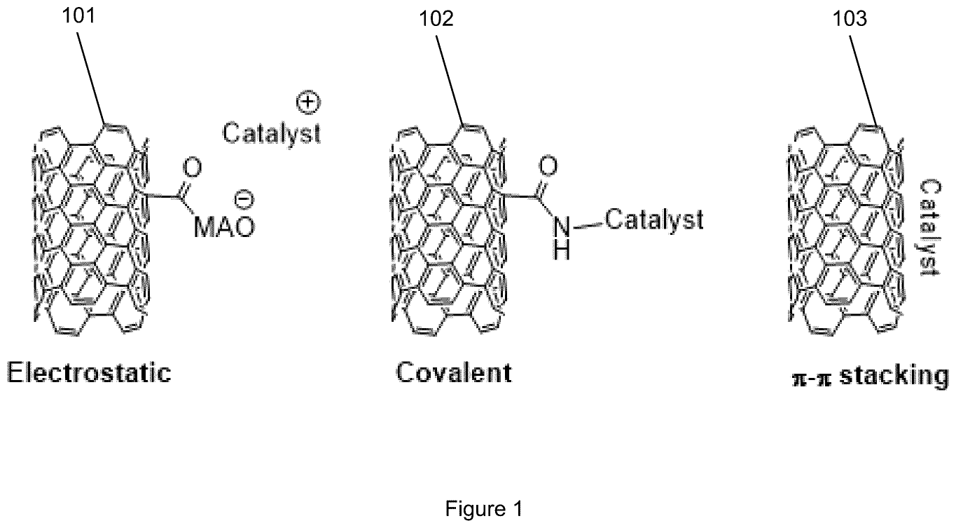

FIG. 1 illustrates various anchoring modes using CNTs support in accordance with an embodiment. 101 shows that a positively charged catalyst can be electrostatically bound with a negatively charged MAO ligand on the CNTs. 102 shows that a catalyst can be covalently bound with the CNTs via an amide bond. 103 shows that a catalyst can be bound with the CNTs via π-π stacking.

Immobilizing catalysts on carbon nanotubes can have various effects on the activity of ethylene polymerization. Factors such as the specific catalyst used, immobilization method, properties of CNTs, and reaction conditions can influence the outcome. For instance, α-diimine nickel catalysts may exhibit decreased ethylene polymerization when immobilized on CNTs. (See, e.g., Talebnezhad, S., et al., Colloid. Polym. Sci., 293, 721-733, 2015; the disclosure of which is incorporated by reference.) However, molecular weight might increase, possibly due to steric effects blocking chain transfer reactions and the preserving role of the macro-ligand MWCNT in preventing site deactivation. On the contrary, a nickel dichloride catalyst with pyridine-imido ligands immobilized on CNTs via an amide linkage may show higher activity for ethylene polymerization than the homogeneous analog. (See, e.g., Zhang, L., et al., Catal. Today, 235, 33-40, 2014; the disclosure of which is incorporated by reference.) Various factors could explain this difference in reactivity, including enhanced dispersion of catalyst particles on the high surface area CNT support, providing more active sites for polymerization. However, decreased activity could result from poor dispersion of the catalyst, electron-donating interfaces of bulky CNT surfaces, blockage of active sites by CNT support, or changes in catalyst structure induced by immobilization.

Previous research has focused on heterogeneous ethylene oligomerization catalysts on CNTS (Xue, J., et al., Chem. Res. Chinese Universities, 38, 552-561, 2022; Li, C., et al., J. Coord. Chem., 73, 1937-1953, 2020; Alshehri, S. M., Adv. Polym. Tech., 35, 21528, 2016; the disclosures of which are incorporated by reference.) Heavy olefins oligomerization with CNT-supported catalysts have not been reported, possibly due to overcrowding around the metal center when anchored on the CNT surface. 1-hexene dimer, primarily used as a plasticizer, can be incorporated into polymers to enhance flexibility, durability, and workability. For instance, adding hexene dimer to polyvinyl chloride (PVC) makes it suitable for applications like flexible PVC films, pipes, wire insulation, flooring, and various other products. However, 1-hexene is challenging to dimerize selectively due to its prone chain length and bulkiness, leading to isomerization reactions. Previously, acid immobilized catalysts like zeolites and MOFs have shown promising activity toward heterogeneous oligomerization of heavy olefins.¹⁸

Many embodiments provide systems and methods for heterogenization on MWCNTs of a pyridine bis-imine iron complex known to dimerize α-olefins into linear internal olefins in homogeneous phase. This selectivity can be explained by an initial 1,2-olefin insertion followed by a 2,1 insertion of the second olefin and chain transfer to produce linear dimers.

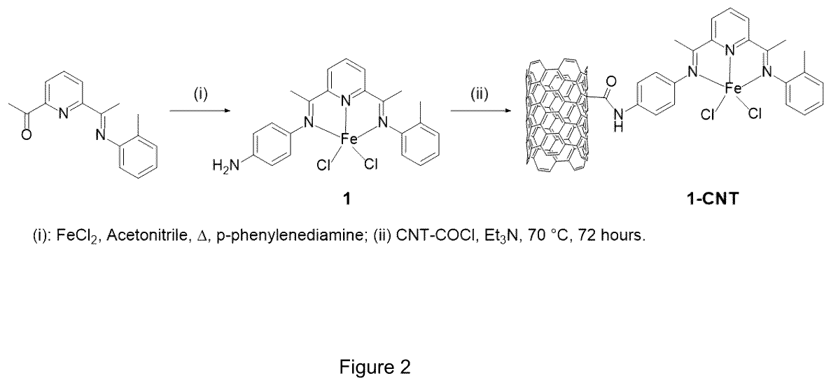

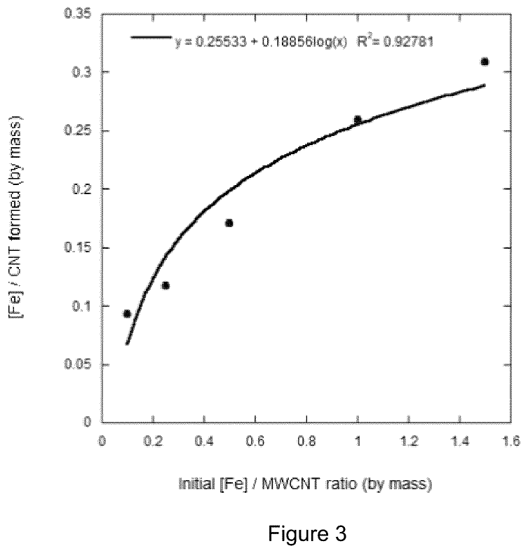

Covalently bonded complexes on CNTs can have higher stability. Many embodiments implement amide linkage to heterogenize the pyridine bis-imine iron complex. Several embodiments synthesize a pyridine bis-imine iron complex with an amino substituent on one of the imine moieties to facilitate complex anchoring. This synthesis can be carried out using an in situ method, where the ketone-imine moiety, 1-{6-[(2-methylphenyl) ethanimidoyl]-2-pyridinyl}-1-ethanone, can be first coordinated to Fe(II), followed by the addition of p-phenylenediamine. FIG. 2 illustrates a synthesis scheme of the heterogenized iron catalyst on CNTs in accordance with an embodiment. Complex 1 is obtained in about 77% yield as a dark green complex. Its purity was measured by Elemental Analysis. The heterogenization process involves the reaction of complex 1 with acyl chloride functionalized MWCNTs (CNT-COCI) for about 72 hours at about 70° C. in the presence of triethylamine. Following thorough washing of the material with acetonitrile, the amount of anchored iron complex is determined using Inductively Coupled Plasma-Optical Emission Spectrometry (ICP-OES). To optimize the loading of metal on the support surface, various ratios of MWCNT/Fe can be investigated. FIG. 3 illustrates catalyst complex loading on MWCNTs in accordance with an embodiment. The amount of iron attached to CNTs increases with an increasing ratio of complex/CNT, eventually reaching a plateau as shown in FIG. 3. Several embodiments use complex and MTCNT ratio of about 1:1 as it offers a favorable balance between loading efficiency (26% of iron complex supported by mass) and complex utilization.

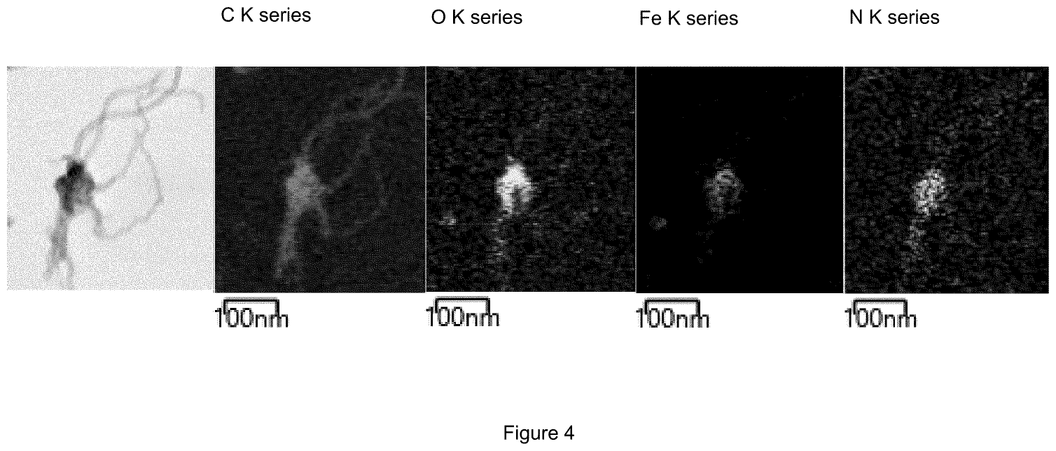

Several embodiments characterize the supported catalyst using TEM/EDS. FIG. 4 illustrates TEM/EDS of immobilized on CNTs in accordance with an embodiment. FIG. 4 shows small broccoli-type agglomeration with high concentration of iron at the CNT surface.

Some embodiments provide characterizations of the homogeneous complex 1 (shown in FIG. 2) and its derivative, complex 2, which features o-tolyl substituents on both imines. Activation of both pre-catalysts occurs through the addition of dry modified methyl aluminoxane (DMMAO) in chlorobenzene followed by 1-hexene introduction at room temperature (about 20-25° C.), with oligomer distribution assessed via Gas Chromatography. Both catalysts exhibit a preference for dimerization. Catalyst 2 demonstrates superior activity, with 99% of 1-hexene consumed within 2 hours, yielding about 81% C12 dimer formation. Catalyst 1 achieves about 67% consumption after about 24 hours, generating about 52-57% C12, indicating slower reactivity compared to non-functionalized catalyst 2. Varying DMMAO equivalents (200, 100 and 50 equiv.) for iron complex activation yields similar outcomes. The presence of an amino substituent on one aryl group of the imines can impact catalyst activity. This behavior could stem from the amino substituent coordinating with the electron-poor metal center on a neighboring complex, hindering 1-hexene coordination, or from DMMAO interacting with the amino group, generating a bulky ligand that limits olefin approach. Furthermore, catalyst 1 displays a much higher isomerization reactivity at the beginning of the process (after about 2 hours), particularly evident with 200 and 100 equivalents of DMMAO, indicating formation of a metal hydride complex probably favored with the presence of the amino group and slow coordination rate of 1-hexene. Table 1 lists 1-hexene dimerization results. Oligomerization conditions: 4.9 mg of catalyst 1 or 2 (19 mg of 1-CNT (representing 4.9 mg of [Fe] complex according to the 26% loading by mass), 1 mL of 1-hexene, 1 mL of chlorobenzene, and DMMAO at room temperature.

TABLE 1
1-Hexene Dimerization

| | | | | | % | % | % |
|---|---|---|---|---|---|---|---|
| | DMMAO | | | 1-hexene | isomers | dimer | trimer |
| Catalyst | equiv. | Time | | consumption | (C6) | (C12) | (C18) |
| 1 | 200 | 2 | h | 12 | 50 | 50 | 0 |
| 1 | 200 | 12 | h | 62 | 24 | 74 | 3 |
| 1 | 200 | 24 | h | 67 | 26 | 52 | 2 |
| 1 | 100 | 2 | h | 23 | 60 | 40 | 0 |
| 1 | 100 | 12 | h | 58 | 27 | 65 | 4 |
| 1 | 100 | 24 | h | 67 | 21 | 57 | 2 |
| 1 | 50 | 2 | h | 22 | 33 | 67 | 0 |
| 1 | 50 | 12 | h | 51 | 30 | 65 | 4 |
| 1 | 50 | 24 | h | 67 | 17 | 57 | 3 |
| 2 | 200 | 2 | h | 99 | 13 | 81 | 4 |
| 2 | 100 | 2 | h | 99 | 27 | 71 | 3 |
| 2 | 50 | 2 | h | 99 | 17 | 81 | 3 |
| 1-CNT | 200 | 2 | h | 70 | 27 | 70 | 5 |
| 1-CNT | 200 | 12 | h | 93 | 32 | 61 | 5 |
| 1-CNT | 200 | 24 | h | 94 | 31 | 65 | 5 |

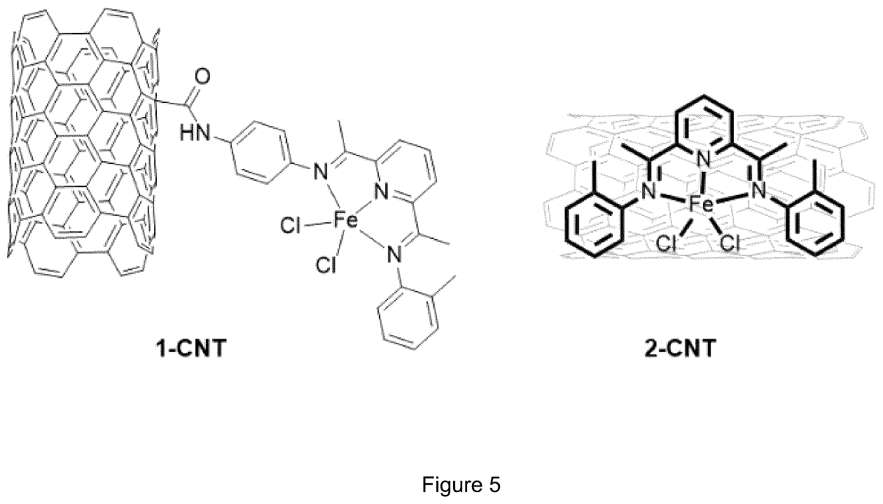

Two heterogenization approaches on MWCNTs are used: IT-IT stacking with catalyst 2 (2-CNT) and covalent bonding via the amide linker for catalyst 1 (1-CNT). FIG. 5 illustrates heterogenized iron catalysts on CNTs in accordance with an embodiment. FIG. 5 shows 1-CNT that uses an amide linker for the catalyst. FIG. 5 shows 2-CNT that uses π-π stacking to attach the attach the catalyst to the CNT. 2-CNT exhibits catalyst leakage during the reaction as evidenced by the purple coloration of the slurry solution, indicating ineffective heterogenization due to weak van der Waals interactions. In contrast, 1-CNT, activated with 200 equivalents of DMMAO, demonstrates efficient performance, achieving about 94% 1-hexene consumption and about 65% selectivity towards C12 dimers after 24 hours. Enhanced activity of 1-CNT over its parent system 1 can be attributed to the involvement of the amino substituent in the amide linkage, preventing interaction with the metal center. 1-CNT can achieve nearly complete olefin consumption within 12 hours. The isomerization process is more pronounced with 1-CNT compared to bis-tolyl catalyst 2 (30% vs. 20% of isomers formation), likely due to increased steric hindrance around the metal center with the CNT support, impeding the approach of a second 1-hexene molecule and therefore favoring the isomerization process.

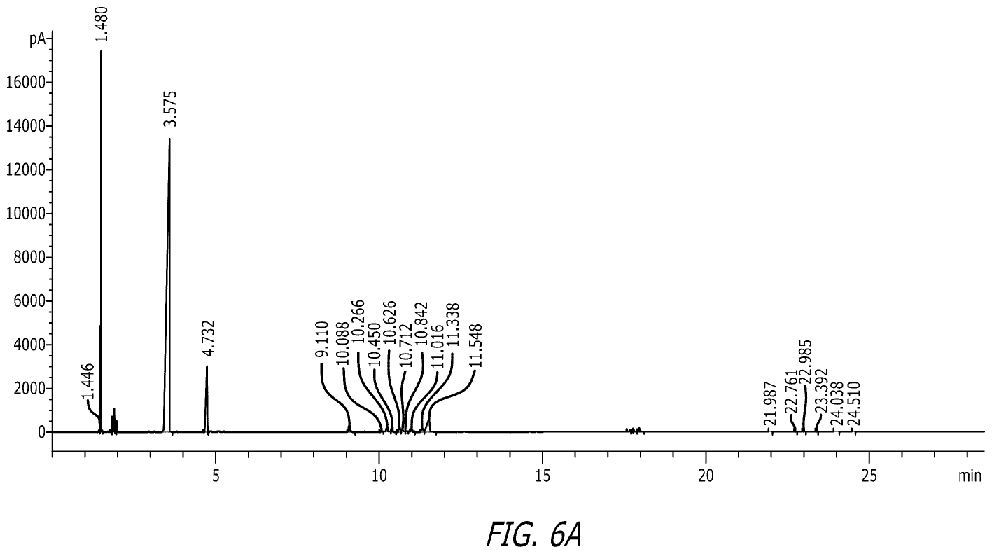

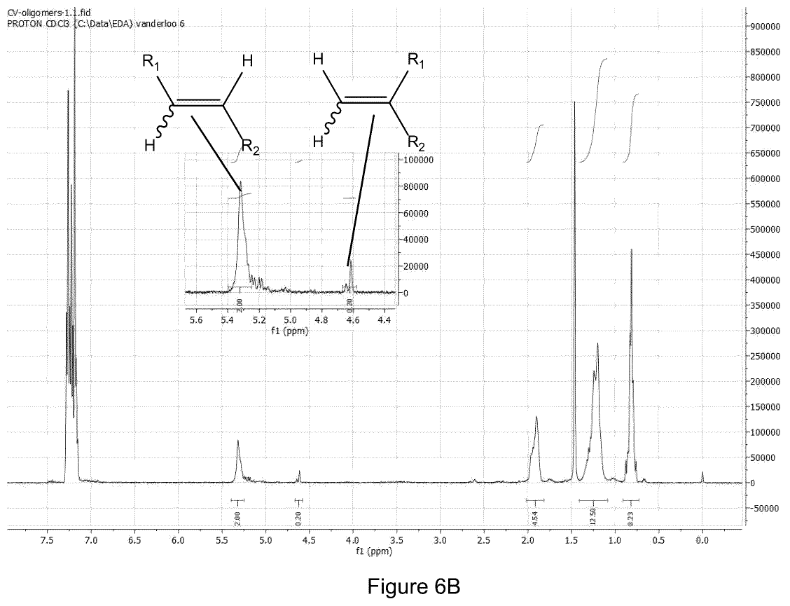

The oligomer obtained from 1-CNT is characterized using 1H NMR spectroscopy and shown in FIG. 6B. The nature of the end-groups in the resulting dimers is examined, revealing the presence of vinylene (R₁CH═CHR₂, δ=5.3 ppm) and vinylidene (H₂C═CR₁R₂, δ=4.6 ppm) resonances in a 90:10 ratio, respectively. This suggests a preference for dimer formation via B-H elimination following 2,1-enchainment of the monomer. The alkyl region (δ=[2.0-0.8 ppm]) integrates perfectly for CH₂C═ (δ=1.9 ppm, 4H), CH₂CH₂CH₂CH₂C═(8=1.2 ppm, 12H), and CH₃ (δ=0.8 ppm, 6H). These findings are consistent with the known mechanism for the homogeneous catalyst, which involves consecutive stages of 1,2-insertion of the initial olefin into the Fe—H bond, followed by 2,1-insertion of the second olefin. Subsequent B—H elimination results in the formation of linear dimers. This result indicates that the CNT does not significantly impede the catalyst's reactivity, allowing the 2,1-approach to still occur.

In some embodiments, a pyridine bis-imine iron complex is supported on MWCNTs and characterized using ICP-OES and TEM/EDS. The amount of complex anchored on the CNTs increases with the loading, eventually reaching a plateau. A 1:1 [Fe]/CNT ratio is optimal, with about 26% of the complex by mass on the CNTs. The 1-CNT complex demonstrated activity in 1-hexene dimerization at room temperature, achieving 94% 1-hexene consumption and 65% selectivity towards C12 dimers. Many embodiments provide heterogenizing a heavy olefin oligomerization catalyst on CNTs, showing that the support does not hinder catalyst activity but rather enhances it compared to the parent complex 1.

### EXEMPLARY EMBODIMENTS

Although specific embodiments of systems and apparatuses are discussed in the following sections, it will be understood that these embodiments are provided as exemplary and are not intended to be limiting.

### Example 1: Experimental Details for CNT-Supported Iron Complex Catalysts for 1-Hexene Dimerization

The experiments are performed using vacuum line and Schlenk tube techniques or in a glovebox under a nitrogen atmosphere unless otherwise noted. The keto-imine ligand, 1-{6-[(2-methylphenyl) ethanimidoyl]-2-pyridinyl}-1-ethanone, is prepared. Carboxylic acid functionalized MWCNTs (MWCNT-COOH) are purchased with an average diameter of 9.5 nm and a length of 1.5 μm (extent of labeling: >8% carboxylic acid functionalized) and functionalized to acyl chloride MWCNTs. Solvents are dried. Anhydrous triethylamine is purchased and used as received. MMAO-12 (7 wt % aluminum in toluene) is purchased and dried at about 60° C. under vacuum to generate a white powder of DMMAO. NMR Spectra are recorded on 400 MHZ spectrometers and referenced to the residual solvent peak. Gas chromatography is performed. Inductively ICP-OES analysis is performed. The TEM-EDS analysis is carried out with a Titan 80-300 TEM. This electron microscope is equipped with an S-TWIN lens. The features are the following: 1) Alignments: 80, 200, 300 kV, 2) Field Emission Gun (X-FEG), 3) Computer-controlled compustage, 4) EDS (Oxford X-MaxN 100TLE 100 mm² SDD for TEM).

For iron complex synthesis. In the glovebox, one equivalent of FeCl₂ (127.0 mg, 1.0 mmol) is added to a solution of keto-imine ligand, 1-{6-[(2,6-dimethylphenyl) ethanimidoyl]-2-pyridinyl}-1-ethanone, (250 mg, 1.0 mmol) in dried acetonitrile (20.0 mL). After heating at about 60° C. for one hour, one equivalent of p-phenylenediamine (108.0 mg, 1.0 mmol) and triethylamine catalyst (0.15 mL) are added. The reaction is heated at about 80° C. overnight. The dark green precipitate is then washed with anhydrous hexanes and dried under vacuum in the glove box (357 mg, 77% yield). Anal. Calcd. for C₂₂H₂₂Cl₂FeN₄ with one molecule of H₂O (due probably to the Elemental Analysis handling): C, 54.89; H, 5.61; N, 11.13. Found: C, 54.82; H, 5.71; N, 11.51.

For synthesizing iron complex supported on MWCNT (1-CNT) for a 1:1 [Fe]/CNT ratio. A chloride functionalized MWCNTs (100.0 mg) and iron catalyst 1 (100.0 mg) are added to a Schlenk tube with dried acetonitrile (15.0 mL) and anhydrous triethylamine (2.25 mL). The reaction is heated at about 70° C. for 72 hours. The black solid is filtered and repeatedly washed with anhydrous acetonitrile until the solvent is colorless, indicating that there is no iron catalyst remaining in the homogenous phase. The product is dried under vacuum, affording a black solid. The amount of CNT is changed according to the [Fe]/CNT ratio. The loading of complex 1 is measured by ICP-OES according to the following ICP digestion method: 20.0 mg of 1-CNT is refluxed in nitric acid (30.0 mL) at about 100° C. overnight. The CNTs are filtered, and the solution is diluted and analyzed by ICP.

For 1-hexene dimerization. In a vial, supported iron complex 1-CNT (20.0 mg) and DMMAO (100 eq, 120.0 mg) are combined. Then, 1-hexene (1.0 mL), and chlorobenzene (1.0 mL) are added after passing them through an alumina plug. The reaction mixture is stirred for about 2, 12 or 24 hours at room temperature. An aliquot of the reaction mixture (0.2 mL) is quenched with toluene (0.4 mL), ethanol (0.1 mL), and three drops of concentrated HCl. A 0.02 g/mL solution of adamantane in toluene (0.4 mL) is added as an internal reference. The mixture is filtered through silica and analyzed by GC. FIG. 6A illustrates GC trace of 1-hexene oligomers obtained by 2 with 100 eq of DMMAO in accordance with an embodiment.

FIG. 6B illustrates 1H NMR in chloroform-di of 1-hexene oligomers obtained by 1-CNT with 100 eq of DMMAO (presence of chlorobenzene) in accordance with an embodiment. The oligomerization reaction is slowly quenched with H₂O. The organic layer is collected, and the volatile oligomers are evaporated. The remaining oligomers are analyzed by NMR spectroscopy in CDCl₃.

FIG. 3 illustrates ICP ratio vs synthetic ratio of iron catalyst 2 to MWCNT in accordance with an embodiment. Acid chloride MWCNT (100 mg) are functionalized with iron catalyst 1 in five different ratios by mass, 1:0.1, 1:0.25, 1:0.5, 1:1, and 1:1.5. 20.0 mg of catalyst complex is refluxed in HNO₃ (30.0 mL) at 100° C. overnight. The CNTs are filtered, and the solution is diluted to be analyzed by ICP.

### Example 2: Experimental Details for CNT-Supported Nickel Complex Catalysts for 1-Pentene Dimerization

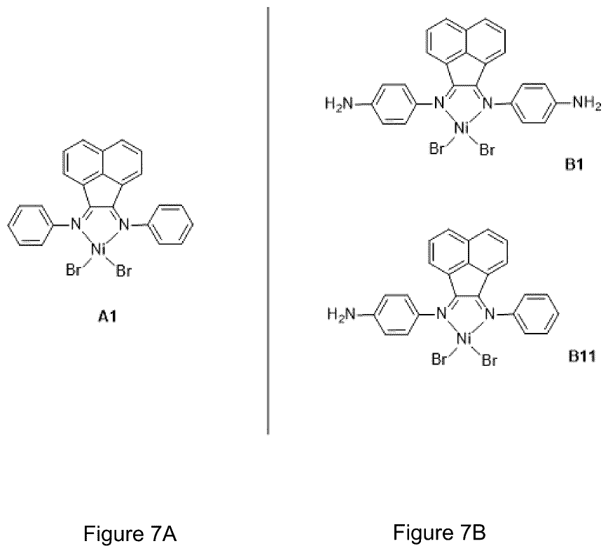

Some embodiments synthesize nickel bearing 1) aryl substituents (A1,) to facilitate interaction with the aromatics on the CNTs (Van der Waals forces), and 2) amino substituents (B1, B11) to covalently bond the complex on the oxidized support by nucleophilic attack (covalent anchoring). FIGS. 7A and 7B illustrate chemical structures of catalysts for heterogenization in accordance with an embodiment. FIG. 7A shows chemical structures of A1 for π-π stacking. FIG. 7B shows chemical structures of B1, and B11 for covalent anchoring.

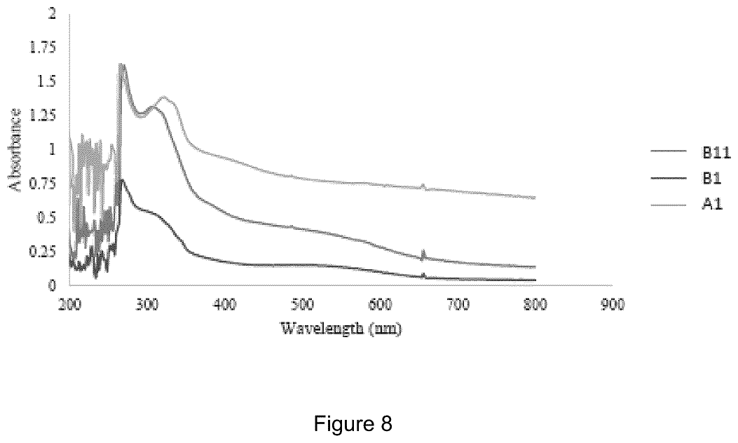

These catalysts are isolated and characterized using Mass Spectrum and UV-Vis. The characterization of the asymmetric systems (B11) is a little bit more challenging (no signal by MS is observed). FIG. 8 illustrates UV-Vis of the nickel complexes A1, B1, and B11.

Some embodiments provide heterogenization of the catalyst complexes on CNTs. For the π-π stacking, catalysts A1 are sonicated in presence of MWCNTs. ICP-AES analysis shows that an averaged 33 wt. % of catalyst is absorbed on the CNTs. For the covalent-bonding strategy: MWCNTs are oxidized with nitric acid and functionalized with complexes B1, and B11 by nucleophilic substitution in presence of an activator (DCC: N,N′-Dicyclohexylcarbodiimide). According to the ICP-AES, this procedure shows an average of 45 wt. % attachment of the complex on CNTs.

Some embodiments provide 1-pentene oligomerization. The parent systems (without CNTs) are first tested towards 1-pentene oligomerization to optimize the conditions. Results are displayed in Table 2. Differences are observed between the pentene consumption and conversion probably due to the evaporation of pentene during the reaction (boiling point: 30° C.). The catalysts supported on CNTs through the π-π stacking approach (A1-CNT) lose their activity towards olefin oligomerization, as expected due to the increase in steric hindrance around the metal center with the proximity of the CNTs (only evaporation of 1-pentene is observed). However, the nickel complex covalently bonded to CNTs shows promising results. The asymmetric system (B11-CNT) seems to have a better activity than the bis-amino analog (B1-CNT), the extra amino substituent could act as an anchor for MMAO (and increase the steric hindrance) or a proton donor. Similar trends are observed with parent complexes (A1 (bis Ph) vs B1 (bis amino).

TABLE 2
1-pentene oligomerization tests at room temperature after 1 hour

| | | Catalyst | | Co- | 1-pentene | 1-pentene | % 2- | % | % heavier |
|---|---|---|---|---|---|---|---|---|---|
| Catalyst | Ligand | loading | | catalyst | consumption | conversion | pentene | C10 | olefins |
| Ni parent | bis | 2 | mg | 300 eq | 77.0% | 44.0% | 45.0% | 53.9% | 1.2% |
| (A1) | Ph | | | MMAO | | | | | |
| Ni parent | bis | 2 | mg | 1000 eq | 70.0% | 39.0% | 42.4% | 53.6% | 3.9% |
| (A1) | Ph | | | MMAO | | | | | |
| Ni parent | bis | 2 | mg | 1000 eq | 73.8% | 16.0% | 0.0% | 100.0% | 0.0% |
| (B1) | amino | | | MMAO | | | | | |
| Ni π-π | bis | 1 | mg | 1000 eq | 72.0% | 0.0% | 0.0% | 0.0% | 0.0% |
| stack | Ph | | | MMAO | | | | | |
| (A1-CNT) | | | | | | | | | |
| Ni CNT | bis | 1.2 | mg | 1000 eq | 70.0% | 6.6% | 85.0% | 4.5% | 0.0% |
| cov (B1- | amino | | | MMAO | | | | | |
| CNT) | | | | | | | | | |
| Ni CNT | Ph | 1.2 | mg | 300 eq | 59.2% | 6.6% | 62.5% | 37.6% | 0.0% |
| cov (B11- | amino | | | MMAO | | | | | |
| CNT) | | | | | | | | | |
| Ni CNT | Ph | 1.2 | mg | 1000 eq | 40.9% | 3.9% | 88.4% | 11.6% | 0.0% |
| cov (B11- | amino | | | MMAO | | | | | |
| CNT) | | | | | | | | | |
| Ni CNT | Ph | 4.8 | mg | 1000 eq | 78.3% | 44.0% | 65.2% | 34.2% | 0.5% |
| cov (B11- | amino | | | MMAO | | | | | |
| CNT) | | | | | | | | | |

Some embodiments provide 1-hexene oligomerization. Covalently attached Ni catalyst has a lower conversion of C6 when compared to the non-CNT system but similar selectivity. π-π stacking Ni catalyst has a similar reactivity as the covalently attached complex.

Table 3 lists the activity of the homogeneous catalysts without CNTs. Nickel catalyst (Ni is active towards C6 oligomerization, however it is less selective towards dimerization (C12), when compared to the Fe-catalyst in example 1.

| | | | | % | | % |
|---|---|---|---|---|---|---|
| Catalyst | Co- | 1-hexene | % | isomers | % | C18- |
| (loading) | catalyst | consumption. | 1-C6 | C6 | C12 | C24 |
| A1 | MMAO- | 84 | 12 | 73 | 27 | 0 |
| (2 mg) | 1000 eq | | | | | |
| A1 | MMAO- | 66 | 32 | 72 | 27 | 0 |
| (2 mg) | 300 eq | | | | | |

Table 4 lists the activity of the covalently attached catalysts on CNTs. Covalently attached Ni catalyst has a lower conversion of C6 when compared to the non-CNT system but similar selectivity.

| | Co- | 1-hexene | % | % isomers | % | % |
|---|---|---|---|---|---|---|
| Catalyst | catalyst | consumption | 1-C6 | C6 | C12 | C18-C24 |
| cov-Ni | MMAO- | 21 | 49 | 83 | 16 | 0 |
| (20 mg) | 1000 eq | | | | | |

### Example 3: Synthesis of Nickel Catalysts

Synthesis of 1,2-Bis(phenylimino) acenaphthene: To a flask containing 50 milliliters of dry toluene, molecular sieves, and 500 milligrams of acenaphthenequinone, 13.9 milligrams of camphorsulfonic acid catalyst are added. An excess of aniline (10 mmol) is added, and the reaction is heated to reflux conditions under nitrogen gas. After 24 hours the reaction is complete, and the sieves are removed by filtrating and washing with toluene. After evaporation, the solid residue is dissolved in 4 mL of chloroform before adding 40 mL of hexanes for recrystallization. After two hours in the fridge, orange crystals precipitated and are subsequently filtered and washed with cold hexanes to yield 601.66 mg of the pure product (65.9%).

¹H NMR (400 MHZ, CDCl₃): δ 7.91 (d, 2H), 7.51 (t, 4H), 7.40 (dd, 4H), 7.16 (d, 4H), 6.85 (d, 2H).

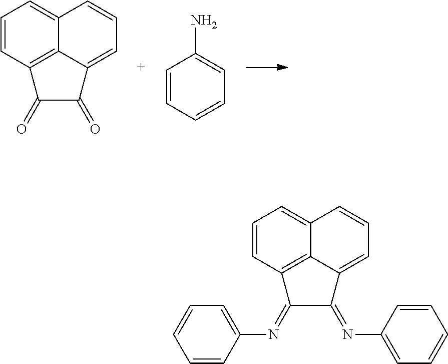 [SMILES: O=C1C(=O)c2cccc3cccc1c23 ; Nc1ccccc1 ; c1ccc(/N=C2/C(=N/c3ccccc3)c3cccc4cccc2c34)cc1]

¹³C NMR (400 MHZ, CDCl₃): δ 161.48, 151.97, 142.07, 131.47, 129.68, 129.19, 128.74, 127.88, 124.64, 124.20, 118.42.

Synthesis of N,N′-1,2-Acenaphthylenediylidenebis [benzenamine-κN] dibromonickel (A1): In a Schlenk flask under nitrogen gas, 217.5 mg of nickel bromide ethylene glycol dimethyl ether is added to a mixture of 1,2-bis(phenylimino) acenaphthene (1.28 equivalents) in 60 mL of dry dichloromethane. The reaction is stirred at room temperature for about 20 hours. Once the stirring is stopped, a brown precipitate is observed. The supernatant is removed before the solid is washed with 3×20 ml of dichloromethane and subsequently dried under vacuum to yield the nickel complex, 388 mg (61.1%).

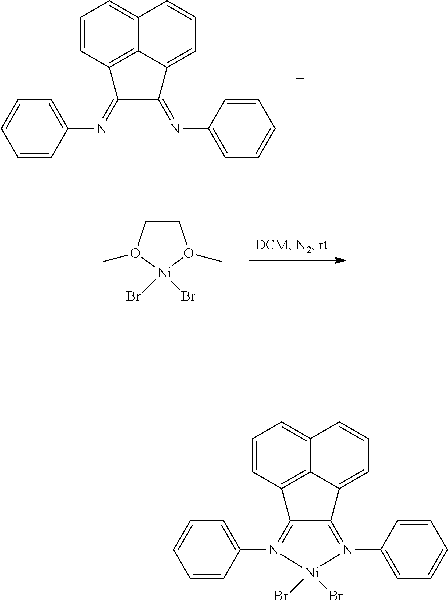

¹H NMR (400 MHZ, CDCl₃): δ 16.84, (J=4.9 Hz), 13.65, (J=6.0 Hz).

Synthesis of 1,2-bis-(p-aminophenylimino)-acenaphthene: A flask is prepared with 500 mg of acenaphthenequinone, 20 mL of dry toluene, and molecular sieves (3 Å) under nitrogen gas. P-phenylenediamine (10 equivalents) is added in excess to avoid formation of a polymer. The mixture is stirred under reflux for 4 hours. Upon completion of the reaction, the sieves are removed through filtration and the flask is washed with acetone to dissolve any remaining product. The solvents are evaporated, and the solid residue is further dried under vacuum. Excess p-phenylenediamine is removed by sublimation, yielding dark purple crystals as the pure product (328 mg, 33.0%).

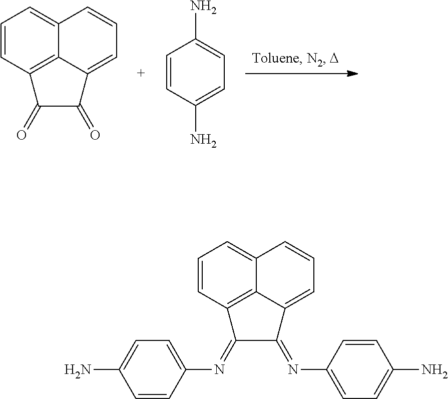 [SMILES: O=C1C(=O)c2cccc3cccc1c23 ; Nc1ccc(N)cc1 ; Nc1ccc(/N=C2/C(=N/c3ccc(N)cc3)c3cccc4cccc2c34)cc1]

¹H NMR (400 MHZ, DMSO): δ 8.02 (d, 2H), 7.51 (dd, 2H), 7.21 (d, 2H), 6.83-6.70 (d, 8H), 5.14 (br, 4H, NH₂).

Synthesis of Nickel, [N¹,N¹-1,2-acenaphthylenediylidenebis [1,4-benzenediamine-κN¹] dibromo complex (B1). To a Shlenk flask containing 200 mg of 1,2-bis-(p-aminophenylimino)-acenaphthene ligand and 1.28 equivalents of nickel bromide ethylene glycol dimethyl ether, 30 mL of dry dichloromethane are added. The reaction is stirred for 20 hours at room temperature. Upon completion, a dark purple precipitate is formed, and the supernatant is removed. The solid is washed with dichloromethane (3×10 mL) to remove any excess ligand. After drying under the vacuum line, the solid measured 195.46 mg (61.0% yield).

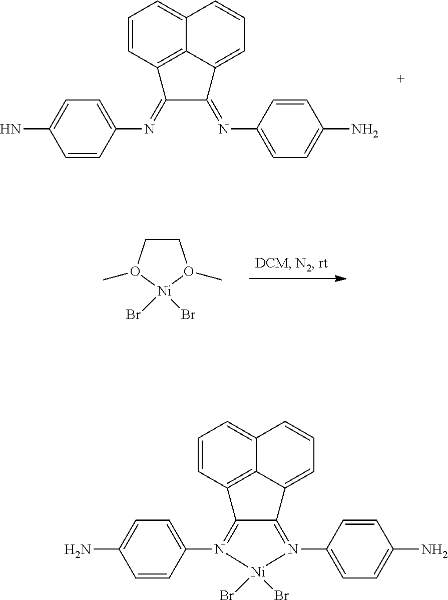

Attaching the nickel π-π stacking catalyst to CNTs. A 50 mg sample of the nickel catalyst (N¹,N¹-1,2-acenaphthylenediylidenebis [1,4-benzenediamine-κN¹] dibromonickel) (A1) is dissolved in 1 mL of THF in a small vial. 5 mg MWCNTs are added to a separate vial with 1 mL of THF. Each vial is sonicated separately for 30 minutes (or until mostly homogenized), before combining the solutions in one vial. The reaction mixture is sonicated for an additional hour. The supernatant is removed, and the mixture of catalyst and MWCNTs are transferred to a centrifuge tube where they are washed repeatedly with DMF until the solvent runs clear. The MWCNTs are washed again twice with 2×20 mL of ether. After drying under vacuum, the CNTs are collected.

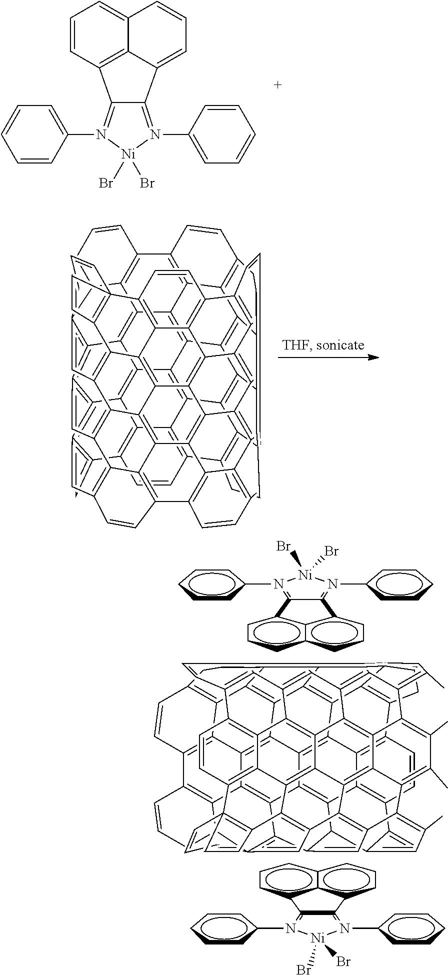

Covalently attaching the nickel catalyst part one: oxidizing MWCNTs. 200 mg of MWCNTs are weighed and added to an Erlenmeyer flask with 100 ml of 70% nitric acid and sonicated for 1 hour. The mixture is transferred to a round bottom flask and

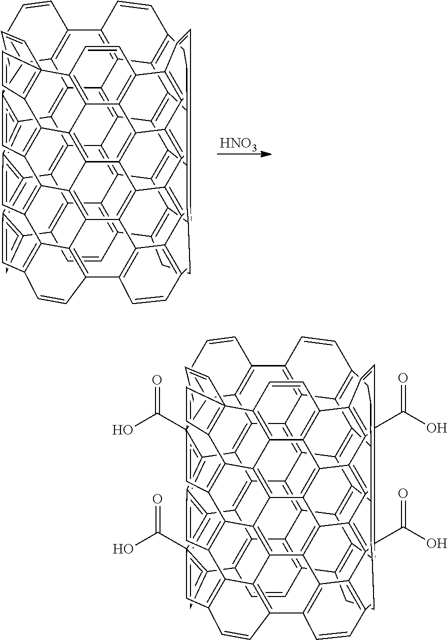

refluxed for an additional hour and a half. Upon completion of the reaction, the MWCNTs are filtered and washed with deionized water until the pH of the washes reaches about 7. The MWCNTs are dried under vacuum and analyzed by infrared spectroscopy.

Covalently attaching the nickel catalyst part two: reaction with nickel catalyst and oxidized MWCNTs. A small portion of the oxidized CNTs (10 mg) are sonicated in 1 mL of dry DMF for one hour. In a Schlenk flask a mixture of 2 mmol of DCC in NMP (1 mL) is added to the sonicated MWCNTs. 200 mg of the covalent nickel catalyst (nickel, N¹, N¹-1,2-acenaphthylenediylidenebis [1,4-benzenediamine-κN¹] dibromo (B1)) is dissolved in 5 mL of dry DMF then added to the CNT using a cannula. The reaction mixture is sonicated for 3 days under nitrogen gas. The CNTs are collected and washed with DMF in centrifuge tubes until the solvent runs clear. The MWCNTs are washed again with ethers to remove and DMF (3×10 mL). The solid residue is collected and dried under a vacuum.

### Example 4: Chromium Catalyst for Heavy Olefin Trimerization

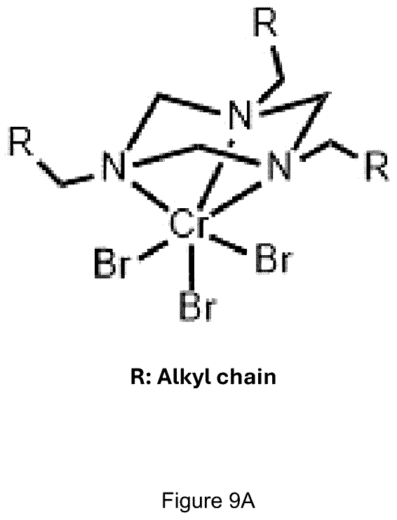

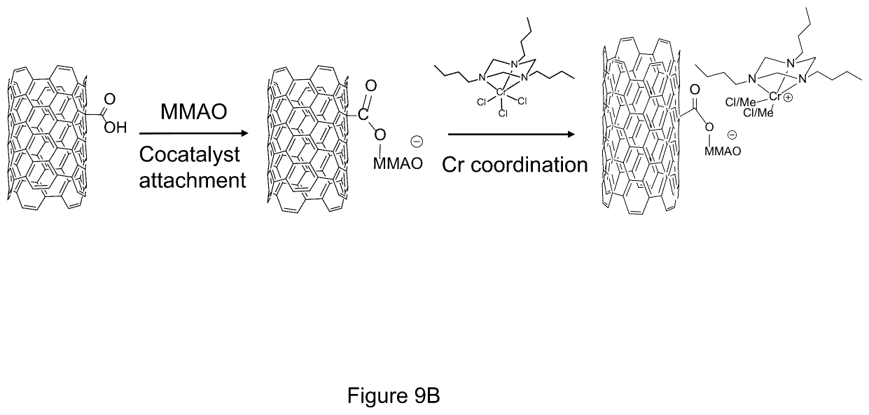

Several embodiments provide chromium (Cr) catalyst complex supported on CNTs for heavy olefin trimerization. FIG. 9A illustrates the chemical structure of the Cr catalyst complex in accordance with an embodiment. FIG. 9B illustrates a schematic of heterogenization of the Cr catalyst complex (especially R=Butyl) in accordance with an embodiment. A co-catalyst MMAO can be attached to a carboxylic bond on the CNTs. The co-catalyst MMAO is negatively charged. The positively charged Cr catalyst complex can be attached to the CNTs via electrostatic bonding. Table 5 lists catalyst activity for trimerization. The results in Table 5 use co-catalyst dry MMAO. The solvent is PhCl. The reaction takes place at room temperature overnight.

| | % hexene | % hexene isomer | % C12 | % C18 |
|---|---|---|---|---|
| Catalyst | consumed | produced | (dimer) | (trimer |
| CNT- | 68.3 | 9.5 | 0 | 18.0 |
| MMAO-Cr | | | | |
| complex | | | | |
| Cr complex | 90.6 | 2.3 | 0 | 40.0 |

### DOCTRINE OF EQUIVALENTS

As can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

As used herein, the singular terms “a,” “an,” and “the,” may include plural referents unless the context clearly dictates otherwise. Reference to an object in the singular is not intended to mean “one and only one” unless explicitly so stated, but rather “one or more.”

As used herein, the terms “approximately,” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

What is claimed is:

1. A catalyst system for alkene oligomerization, comprising:
a catalyst complex supported on a plurality of carbon nanotubes (CNTs), wherein the catalyst complex comprises a transition metal;
wherein the catalyst system catalyzes an alkene oligomerization reaction, wherein an alkene with at least 3 carbon atoms is polymerized to form a product such that a number of carbon atoms of the product is at least doubled.

2. The catalyst system of claim 1, wherein the transition metal is selected from the group consisting of: iron, nickel, and chromium.

3. The catalyst system of claim 1, wherein at least one of the plurality of CNTs is a single-walled carbon nanotube or a multi-walled carbon nanotube.

4. The catalyst system of claim 1, wherein the alkene is selected from the group consisting of: propylene, 1-butene, 2-butene, isobutylene, 1-pentene, 2-pentene, 2-methyl-1-butene, 3-methyl-1-butene, 2-methyl-2-butene, 1-hexene, 2-hexene, 3-hexene, 2-methyl-1-pentene, 3-methyl-1-pentene, 4-methyl-1-pentene, 2-methyl-2-pentene, 3-methyl-2-pentene, 4-methyl-2-pentene, 2,3-dimethyl-1-butene, 3,3-dimethyl-1-butene, 2,3-dimethyl-2-butene, and 2-ethyl-1-butene.

5. The catalyst system of claim 1, wherein the alkene oligomerization is a dimerization reaction, and the number of carbon atoms of the product is doubled.

6. The catalyst system of claim 1, wherein the alkene oligomerization is a trimerization reaction, and the number of carbon atoms of the product is tripled.

7. The catalyst system of claim 1, wherein the catalyst complex is supported on a plurality of CNTs via a method selected from the group consisting of: a covalent bond, an electrostatic bond, and π-π stacking.

8. The catalyst system of claim 1, wherein the catalyst complex forms at least one amid bond with at least one of the plurality of CNTs.

9. The catalyst system of claim 1, further comprising a co-catalyst.

10. The catalyst system of claim 9, wherein the co-catalyst is selected from the group consisting of: methyl aluminoxane, modified methyl aluminoxane, diethylaluminium chloride (Et2AlCl), and EtAlCl2.

11. The catalyst system of claim 1, wherein the alkene is 1-hexene, the catalyst complex comprises iron, and the product has 12 carbon atoms.

12. The catalyst system of claim 1, wherein the product is configured to be a portion of jet fuel.

13. A method for alkene oligomerization, comprising:
catalyzing an alkene oligomerization reaction with a catalyst system comprising a catalyst complex supported on a plurality of carbon nanotubes (CNTs), wherein an alkene with at least 3 carbon atoms is polymerized to a product such that a number of carbon atoms of the product is at least doubled.

14. The method of claim 13, wherein the catalyst complex comprises a transition metal.

15. The method of claim 14, wherein the transition metal is selected from the group consisting of: iron, nickel, and chromium.

16. The method of claim 13, wherein at least one of the plurality of CNTs is a single-walled carbon nanotube or a multi-walled carbon nanotube.

17. The method of claim 13, wherein the alkene is selected from the group consisting of: propylene, 1-butene, 2-butene, isobutylene, 1-pentene, 2-pentene, 2-methyl-1-butene, 3-methyl-1-butene, 2-methyl-2-butene, 1-hexene, 2-hexene, 3-hexene, 2-methyl-1-pentene, 3-methyl-1-pentene, 4-methyl-1-pentene, 2-methyl-2-pentene, 3-methyl-2-pentene, 4-methyl-2-pentene, 2,3-dimethyl-1-butene, 3,3-dimethyl-1-butene, 2,3-dimethyl-2-butene, and 2-ethyl-1-butene.

18. The method of claim 13, wherein the alkene oligomerization is a dimerization reaction, and the number of carbon atoms of the product is doubled.

19. The method of claim 13, wherein the alkene oligomerization is a trimerization reaction, and the number of carbon atoms of the product is tripled.

20. The method of claim 13, wherein the catalyst complex is supported on a plurality of CNTs via a method selected from the group consisting of: a covalent bond, an electrostatic bond, and IT-IT stacking.

21. The method of claim 13, wherein the catalyst complex forms at least one amid bond with at least one of the plurality of CNTs.

22. The method of claim 13, wherein the catalyst system further comprises a co-catalyst.

23. The method of claim 22, wherein the co-catalyst is selected from the group consisting of: methyl aluminoxane, modified methyl aluminoxane, diethylaluminium chloride (Et2AlCl), and EtAlCl2.

24. The method of claim 13, wherein the alkene is 1-hexene, the catalyst complex comprises iron, and the product has 12 carbon atoms.

25. The method of claim 13, wherein the product is configured to be a portion of jet fuel.
